(19) United States
(12) Patent Application Publication
(10) Pub. No.: US 2026/0158209 A1
(43) Pub. Date: Jun. 11, 2026

(54) LIQUID-COLLECTION CANISTER WITH MULTI-ORIENTATION FILTER

(71) Applicant: KCI Manufacturing Unlimited Company, Westmeath (IE)

(72) Inventors: Benjamin A. PRATT, Poole (GB); James K. SEDDON, Wimborne (GB)

(21) Appl. No.: 18/706,436

(22) PCT Filed: Sep. 26, 2022
(86) PCT No.: PCT/IB2022/059120

§ 371 Date: May 1, 2024

Related U.S. Application Data
(60) Provisional application No. 63/263,613, filed on Nov. 5, 2021.

(51) Int. Cl. A61M 1/00

(52) U.S. Cl. CPC A61M 1/984; A61M 1/784; A61M 1/966; A61M 2205/21; A61M 2205/3344; A61M 2205/7536

(57) ABSTRACT

A liquid-collection canister for collecting liquid from a tissue site to which reduced pressure treatment is applied. The canister may include a canister body, a canister lid, a filter carrier, and a filter. The canister lid may define a liquid collection chamber with the canister body. The liquid collection chamber may include one or more internal walls configured to collect and be exposed to the liquid from the tissue site. The filter carrier may be suspended within the liquid collection chamber and separated from the internal walls. The filter may be coupled to the filter carrier and configured to allow gaseous communication with the liquid collection chamber and to prevent liquid from leaving the liquid collection chamber.

### CROSS-REFERENCE TO RELATED APPLICATIONS

This application is a U.S. National Stage Entry of PCT/IB2022/059120, filed Sep. 26, 2022, which claims the benefit, under 35 U.S.C. § 119(e), of U.S. Provisional Application No. 63/263,613, filed on Nov. 5, 2021, each of which are incorporated herein by reference in its entirety.

### TECHNICAL FIELD

The invention set forth in the appended claims relates generally to tissue treatment systems and more particularly, but without limitation, to a reduced-pressure, liquid-collection canister having a filter that allows operation of the canister in multiple orientations.

### BACKGROUND

Clinical studies and practice have shown that reducing pressure in proximity to a tissue site can augment and accelerate growth of new tissue at the tissue site. The applications of this phenomenon are numerous, but it has proven particularly advantageous for treating wounds. Regardless of the etiology of a wound, whether trauma, surgery, or another cause, proper care of the wound is important to the outcome. Treatment of wounds or other tissue with reduced pressure may be commonly referred to as “negative-pressure therapy,” but is also known by other names, including “negative-pressure wound therapy,” “reduced-pressure therapy,” “vacuum therapy,” “vacuum-assisted closure,” and “topical negative-pressure,” for example. Negative-pressure therapy may provide a number of benefits, including migration of epithelial and subcutaneous tissues, improved blood flow, and micro-deformation of tissue at a wound site. Together, these benefits can increase development of granulation tissue and reduce healing times.

While the clinical benefits of negative-pressure therapy are widely known, improvements to therapy systems, components, and processes may benefit healthcare providers and patients.

### BRIEF SUMMARY

New and useful systems, apparatuses, and methods for collecting liquid from a tissue site in a negative-pressure therapy environment are set forth in the appended claims. Illustrative example embodiments are also provided to enable a person skilled in the art to make and use the claimed subject matter.

For example, in some example embodiments, a liquid-collection canister for collecting liquid from a tissue site to which reduced pressure treatment is applied includes a canister body, a canister lid, a filter carrier, and a filter. The canister lid defines a liquid collection chamber with the canister body. The liquid collection chamber includes one or more internal walls configured to collect and be exposed to the liquid from the tissue site. The filter carrier is suspended within the liquid collection chamber and separated from the internal walls. The filter is coupled to the filter carrier and configured to allow gaseous communication with the liquid collection chamber and to prevent liquid from leaving the liquid collection chamber.

In another illustrative example embodiment, a liquid-collection canister for collecting liquid from a tissue site includes a liquid collection chamber and a filter assembly. The liquid collection chamber is configured to collect the liquid from the tissue site. The filter assembly is configured to be positioned within the liquid collection chamber in fluid communication between the liquid collection chamber and a reduced pressure port. At least a portion of the filter assembly may also be configured to reach opposite and transverse ends of the liquid collection chamber.

In yet another example embodiment, a system for collecting liquid from a tissue site to which reduced pressure treatment is applied includes a liquid collection chamber having a canister body, a canister lid, a filter carrier, and a filter. The canister body includes a collection port and a first sensing port. The canister lid is configured to define a liquid collection chamber relative to the canister body and includes a reduced pressure port and a second sensing port. The second sensing port is configured to be fluidly coupled to the first sensing port. The filter carrier is configured to be coupled to the reduced pressure port and extend across the liquid collection chamber. The filter is coupled to the filter carrier and configured to allow gaseous communication and to prevent liquid from leaving the canister body. The system further includes a dressing fluidly coupled to the collection port and the first sensing port. The system also includes a reduced pressure source fluidly coupled to the reduced pressure port.

Objectives, advantages, and a preferred mode of making and using the claimed subject matter may be understood best by reference to the accompanying drawings in conjunction with the following detailed description of illustrative embodiments.

### BRIEF DESCRIPTION OF THE DRAWINGS

FIG. 1 is a block diagram of an example embodiment of a therapy system that can provide negative-pressure treatment in accordance with this specification;

FIG. 2 is an exploded view of an example embodiment of a canister that may be associated with some embodiments of the therapy system of FIG. 1;

FIG. 3A is a top view of the canister of FIG. 2, illustrating additional details that may be associated with some embodiments;

FIG. 3B is a side view of the canister of FIG. 2, illustrating additional details that may be associated with some embodiments;

FIG. 3C is another side view of the canister of FIG. 2, illustrating additional details that may be associated with some embodiments;

FIG. 4 is another exploded view of the canister of FIG. 2, illustrating additional details that may be associated with some embodiments;

FIG. 5 is a section view of the canister of FIG. 2, illustrating additional details that may be associated with some embodiments;

FIG. 6 is a cross-section view of the canister of FIG. 2, illustrating additional details that may be associated with some embodiments;

FIG. 7 is an exploded view of an example embodiment of a filter assembly that may be associated with some embodiments of the canister of FIG. 2;

FIG. 8 is an exploded view of another example embodiment of a filter assembly that may be associated with some embodiments of the canister of FIG. 2;

FIG. 9 is an exploded view of another example embodiment of a filter assembly that may be associated with some embodiments of the canister of FIG. 2;

FIG. 10 is a perspective view of another example embodiment of a filter assembly that may be associated with some embodiments of the canister of FIG. 2;

FIG. 11A is a perspective view of another example embodiment of a filter assembly that may be associated with some embodiments of the canister of FIG. 2;

FIG. 11B is a section view of the filter assembly of FIG. 11A, illustrating additional details that may be associated with some embodiments;

FIG. 12A is a perspective view of another example embodiment of a filter assembly that may be associated with some embodiments of the canister of FIG. 2;

FIG. 12B is a section view of the filter assembly of FIG. 12A, illustrating additional details that may be associated with some embodiments;

FIG. 13A is a top view of another example embodiment of a canister that may be associated with some embodiments of the therapy system of FIG. 1;

FIG. 13B is a side view of the canister of FIG. 13A, illustrating additional details that may be associated with some embodiments;

FIG. 13B is another side view of the canister of FIG. 13A, illustrating additional details that may be associated with some embodiments;

FIG. 14 is an exploded view of another example embodiment of a filter assembly and a lid of a canister that may be associated with some embodiments of the therapy system of FIG. 1;

FIG. 15 is an exploded view of another example embodiment of a filter assembly and a lid of a canister that may be associated with some embodiments of the therapy system of FIG. 1; and

FIG. 16 is an exploded view of yet another example embodiment of a filter assembly and a lid of a canister that may be associated with some embodiments of the therapy system of FIG. 1.

### DESCRIPTION OF EXAMPLE EMBODIMENTS

The following description of example embodiments provides information that enables a person skilled in the art to make and use the subject matter set forth in the appended claims, but it may omit certain details already well-known in the art. The following detailed description is, therefore, to be taken as illustrative and not limiting.

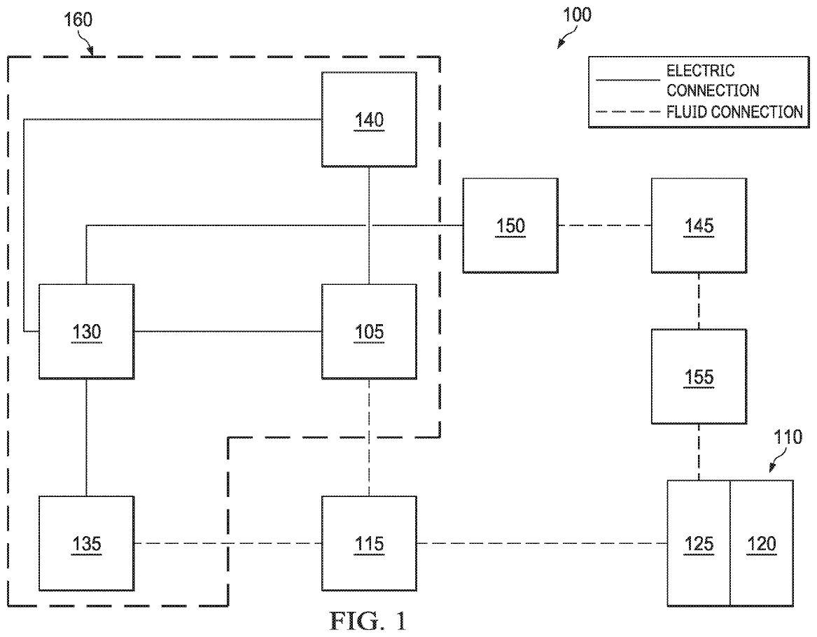

FIG. 1 is a block diagram of an example embodiment of a therapy system 100 that can provide negative-pressure therapy with instillation of topical treatment solutions to a tissue site in accordance with this specification.

The term “tissue site” in this context broadly refers to a wound, defect, or other treatment target located on or within tissue, including, but not limited to, bone tissue, adipose tissue, muscle tissue, neural tissue, dermal tissue, vascular tissue, connective tissue, cartilage, tendons, or ligaments. A wound may include chronic, acute, traumatic, subacute, and dehisced wounds, partial-thickness burns, ulcers (such as diabetic, pressure, or venous insufficiency ulcers), flaps, and grafts, for example. The term “tissue site” may also refer to areas of any tissue that are not necessarily wounded or defective, but are instead areas in which it may be desirable to add or promote the growth of additional tissue. For example, negative pressure may be applied to a tissue site to grow additional tissue that may be harvested and transplanted.

The therapy system 100 may include a source or supply of negative pressure, such as a negative-pressure source 105, and one or more distribution components. A distribution component is preferably detachable and may be disposable, reusable, or recyclable. A dressing, such as a dressing 110, and a fluid container or canister, such as a canister 115, are examples of distribution components that may be associated with some examples of the therapy system 100. As illustrated in the example of FIG. 1, the dressing 110 may comprise or consist essentially of a tissue interface 120, a cover 125, or both in some embodiments.

A fluid conductor is another illustrative example of a distribution component. A “fluid conductor,” in this context, broadly includes a tube, pipe, hose, conduit, or other structure with one or more lumina or open pathways adapted to convey a fluid between two ends. Typically, a tube is an elongated, cylindrical structure with some flexibility, but the geometry and rigidity may vary. Moreover, some fluid conductors may be molded into or otherwise integrally combined with other components. Distribution components may also include or comprise interfaces or fluid ports to facilitate coupling and de-coupling other components. In some embodiments, for example, a dressing interface may facilitate coupling a fluid conductor to the dressing 110. For example, such a dressing interface may be a SENSAT.R.A.C.™ Pad available from Kinetic Concepts, Inc. of San Antonio, Texas.

The therapy system 100 may also include a regulator or controller, such as a controller 130. Additionally, the therapy system 100 may include sensors to measure operating parameters and provide feedback signals to the controller 130 indicative of the operating parameters. As illustrated in FIG. 1, for example, the therapy system 100 may include a first sensor 135 and a second sensor 140 coupled to the controller 130.

The therapy system 100 may also include a source of instillation solution. For example, a solution source 145 may be fluidly coupled to the dressing 110, as illustrated in the example embodiment of FIG. 1. The solution source 145 may be fluidly coupled to a positive-pressure source such as a positive-pressure source 150, a negative-pressure source such as the negative-pressure source 105, or both in some embodiments. A regulator, such as an instillation regulator 155, may also be fluidly coupled to the solution source 145 and the dressing 110 to ensure proper dosage of instillation solution (e.g. saline) to a tissue site. For example, the instillation regulator 155 may comprise a piston that can be pneumatically actuated by the negative-pressure source 105 to draw instillation solution from the solution source during a negative-pressure interval and to instill the solution to a dressing during a venting interval. Additionally or alternatively, the controller 130 may be coupled to the negative-pressure source 105, the positive-pressure source 150, or both, to control dosage of instillation solution to a tissue site. In some embodiments, the instillation regulator 155 may also be fluidly coupled to the negative-pressure source 105 through the dressing 110, as illustrated in the example of FIG. 1.

Some components of the therapy system 100 may be housed within or used in conjunction with other components, such as sensors, processing units, alarm indicators, memory, databases, software, display devices, or user interfaces that further facilitate therapy. For example, in some embodiments, the negative-pressure source 105 may be combined with the controller 130 and other components into a therapy unit 160.

In general, components of the therapy system 100 may be coupled directly or indirectly. For example, the negative-pressure source 105 may be directly coupled to the canister 115 and may be indirectly coupled to the dressing 110 through the canister 115. Coupling may include fluid, mechanical, thermal, electrical, or chemical coupling (such as a chemical bond), or some combination of coupling in some contexts. For example, the negative-pressure source 105 may be electrically coupled to the controller 130 and may be fluidly coupled to one or more distribution components to provide a fluid path to a tissue site. In some embodiments, components may also be coupled by virtue of physical proximity, being integral to a single structure, or being formed from the same piece of material.

A negative-pressure supply, such as the negative-pressure source 105, may be a reservoir of air at a negative pressure or may be a manual or electrically-powered device, such as a vacuum pump, a suction pump, a wall suction port available at many healthcare facilities, or a micro-pump, for example. “Negative pressure” generally refers to a pressure less than a local ambient pressure, such as the ambient pressure in a local environment external to a sealed therapeutic environment. In many cases, the local ambient pressure may also be the atmospheric pressure at which a tissue site is located. Alternatively, the pressure may be less than a hydrostatic pressure associated with tissue at the tissue site. Unless otherwise indicated, values of pressure stated herein are gauge pressures. References to increases in negative pressure typically refer to a decrease in absolute pressure, while decreases in negative pressure typically refer to an increase in absolute pressure. While the amount and nature of negative pressure provided by the negative-pressure source 105 may vary according to therapeutic requirements, the pressure is generally a low vacuum, also commonly referred to as a rough vacuum, between −5 mm Hg (−667 Pa) and −500 mm Hg (−66.7 kPa). Common therapeutic ranges are between −50 mm Hg (−6.7 kPa) and −300 mm Hg (−39.9 kPa).

The canister 115 is representative of a container, canister, pouch, or other storage component, which can be used to manage exudates and other fluids withdrawn from a tissue site. In many environments, a rigid container may be preferred or required for collecting, storing, and disposing of fluids. In other environments, fluids may be properly disposed of without rigid container storage, and a re-usable container could reduce waste and costs associated with negative-pressure therapy.

A controller, such as the controller 130, may be a microprocessor or computer programmed to operate one or more components of the therapy system 100, such as the negative-pressure source 105. In some embodiments, for example, the controller 130 may be a microcontroller, which generally comprises an integrated circuit containing a processor core and a memory programmed to directly or indirectly control one or more operating parameters of the therapy system 100. Operating parameters may include the power applied to the negative-pressure source 105, the pressure generated by the negative-pressure source 105, or the pressure distributed to the tissue interface 120, for example. The controller 130 is also preferably configured to receive one or more input signals, such as a feedback signal, and programmed to modify one or more operating parameters based on the input signals.

Sensors, such as the first sensor 135 and the second sensor 140, may be any apparatus operable to detect or measure a physical phenomenon or property, and generally provide a signal indicative of the phenomenon or property that is detected or measured. For example, the first sensor 135 and the second sensor 140 may be configured to measure one or more operating parameters of the therapy system 100. In some embodiments, the first sensor 135 may be a transducer configured to measure pressure in a pneumatic pathway and convert the measurement to a signal indicative of the pressure measured. In some embodiments, for example, the first sensor 135 may be a piezo-resistive strain gauge. The second sensor 140 may optionally measure operating parameters of the negative-pressure source 105, such as a voltage or current, in some embodiments. Preferably, the signals from the first sensor 135 and the second sensor 140 are suitable as an input signal to the controller 130, but some signal conditioning may be appropriate in some embodiments. For example, the signal may need to be filtered or amplified before it can be processed by the controller 130. Typically, the signal is an electrical signal, but may be represented in other forms, such as an optical signal.

The tissue interface 120 can be generally adapted to partially or fully contact a tissue site. The tissue interface 120 may take many forms, and may have many sizes, shapes, or thicknesses, depending on a variety of factors, such as the type of treatment being implemented or the nature and size of a tissue site. For example, the size and shape of the tissue interface 120 may be adapted to the contours of deep and irregular shaped tissue sites. Any or all of the surfaces of the tissue interface 120 may have an uneven, coarse, or jagged profile.

In some embodiments, the tissue interface 120 may comprise or consist essentially of a manifold. A manifold in this context may comprise or consist essentially of a means for collecting or distributing fluid across or through the tissue interface 120 under pressure. For example, a manifold may be adapted to receive negative pressure from a source and distribute negative pressure through multiple apertures across or through the tissue interface 120, which may have the effect of collecting fluid from a tissue site and drawing the fluid toward the source. In some embodiments, the fluid path may be reversed or a secondary fluid path may be provided to facilitate delivering fluid, such as fluid from a source of instillation solution, to a tissue site.

In some illustrative embodiments, a manifold may comprise a plurality of pathways, which can be interconnected to improve distribution or collection of fluids. In some illustrative embodiments, a manifold may comprise or consist essentially of a porous material having interconnected fluid pathways. Examples of suitable porous material that can be adapted to form interconnected fluid pathways (e.g., channels) may include cellular foam, including open-cell foam such as reticulated foam; porous tissue collections; and other porous material such as gauze or felted mat that generally include pores, edges, and/or walls. Liquids, gels, and other foams may also include or be cured to include apertures and fluid pathways. In some embodiments, a manifold may additionally or alternatively comprise projections that form interconnected fluid pathways. For example, a manifold may be molded to provide surface projections that define interconnected fluid pathways.

In some embodiments, the tissue interface 120 may comprise or consist essentially of reticulated foam having pore sizes and free volume that may vary according to needs of a prescribed therapy. For example, reticulated foam having a free volume of at least 90% may be suitable for many therapy applications, and foam having an average pore size in a range of 400-600 microns (40-50 pores per inch) may be particularly suitable for some types of therapy. The tensile strength of the tissue interface 120 may also vary according to needs of a prescribed therapy. For example, the tensile strength of foam may be increased for instillation of topical treatment solutions. The 25% compression load deflection of the tissue interface 120 may be at least 0.35 pounds per square inch, and the 65% compression load deflection may be at least 0.43 pounds per square inch. In some embodiments, the tensile strength of the tissue interface 120 may be at least 10 pounds per square inch. The tissue interface 120 may have a tear strength of at least 2.5 pounds per inch. In some embodiments, the tissue interface may be foam comprised of polyols such as polyester or polyether, isocyanate such as toluene diisocyanate, and polymerization modifiers such as amines and tin compounds. In some examples, the tissue interface 120 may be reticulated polyurethane foam such as found in GRANUFOAM™ dressing or V.A.C. VERAFLO™ dressing, both available from Kinetic Concepts, Inc. of San Antonio, Texas.

The thickness of the tissue interface 120 may also vary according to needs of a prescribed therapy. For example, the thickness of the tissue interface may be decreased to reduce tension on peripheral tissue. The thickness of the tissue interface 120 can also affect the conformability of the tissue interface 120. In some embodiments, a thickness in a range of about 5 millimeters to 10 millimeters may be suitable.

The tissue interface 120 may be either hydrophobic or hydrophilic. In an example in which the tissue interface 120 may be hydrophilic, the tissue interface 120 may also wick fluid away from a tissue site, while continuing to distribute negative pressure to the tissue site. The wicking properties of the tissue interface 120 may draw fluid away from a tissue site by capillary flow or other wicking mechanisms. An example of a hydrophilic material that may be suitable is a polyvinyl alcohol, open-cell foam such as V.A.C. WHITEFOAM™ dressing available from Kinetic Concepts, Inc. of San Antonio, Texas. Other hydrophilic foams may include those made from polyether. Other foams that may exhibit hydrophilic characteristics include hydrophobic foams that have been treated or coated to provide hydrophilicity.

In some embodiments, the tissue interface 120 may be constructed from bioresorbable materials. Suitable bioresorbable materials may include, without limitation, a polymeric blend of polylactic acid (PLA) and polyglycolic acid (PGA). The polymeric blend may also include, without limitation, polycarbonates, polyfumarates, and capralactones. The tissue interface 120 may further serve as a scaffold for new cell-growth, or a scaffold material may be used in conjunction with the tissue interface 120 to promote cell-growth. A scaffold is generally a substance or structure used to enhance or promote the growth of cells or formation of tissue, such as a three-dimensional porous structure that provides a template for cell growth. Illustrative examples of scaffold materials include calcium phosphate, collagen, PLA/PGA, coral hydroxy apatites, carbonates, or processed allograft materials.

In some embodiments, the cover 125 may provide a bacterial barrier and protection from physical trauma. The cover 125 may also be constructed from a material that can reduce evaporative losses and provide a fluid seal between two components or two environments, such as between a therapeutic environment and a local external environment. The cover 125 may comprise or consist of, for example, an elastomeric film or membrane that can provide a seal adequate to maintain a negative pressure at a tissue site for a given negative-pressure source. The cover 125 may have a high moisture-vapor transmission rate (MVTR) in some applications. For example, the MVTR may be at least 250 grams per square meter per twenty-four hours in some embodiments, measured using an upright cup technique according to ASTM E96/E96M Upright Cup Method at 38° C. and 10% relative humidity (RH). In some embodiments, an MVTR up to 5,000 grams per square meter per twenty-four hours may provide effective breathability and mechanical properties.

In some example embodiments, the cover 125 may be a polymer drape, such as a polyurethane film, that is permeable to water vapor but impermeable to liquid. Such drapes typically have a thickness in the range of 25-50 microns. For permeable materials, the permeability generally should be low enough that a desired negative pressure may be maintained. The cover 125 may comprise, for example, one or more of the following materials: polyurethane (PU), such as hydrophilic polyurethane; cellulosics; hydrophilic polyamides; polyvinyl alcohol; polyvinyl pyrrolidone; hydrophilic acrylics; silicones, such as hydrophilic silicone elastomers; natural rubbers; polyisoprene; styrene butadiene rubber; chloroprene rubber; polybutadiene; nitrile rubber; butyl rubber; ethylene propylene rubber; ethylene propylene diene monomer; chlorosulfonated polyethylene; polysulfide rubber; ethylene vinyl acetate (EVA); co-polyester; and polyether block polymide copolymers. Such materials are commercially available as, for example, Tegaderm® drape, commercially available from 3M Company, Minneapolis Minnesota; polyurethane (PU) drape, commercially available from Avery Dennison Corporation, Pasadena, California; polyether block polyamide copolymer (PEBAX), for example, from Arkema S.A., Colombes, France; and Inspire 2301 and Inpsire 2327 polyurethane films, commercially available from Expopack Advanced Coatings, Wrexham, United Kingdom. In some embodiments, the cover 125 may comprise INSPIRE 2301 having an MVTR (upright cup technique) of 2600 g/m²/24 hours and a thickness of about 30 microns.

An attachment device may be used to attach the cover 125 to an attachment surface, such as undamaged epidermis, a gasket, or another cover. The attachment device may take many forms. For example, an attachment device may be a medically-acceptable, pressure-sensitive adhesive configured to bond the cover 125 to epidermis around a tissue site. In some embodiments, for example, some or all of the cover 125 may be coated with an adhesive, such as an acrylic adhesive, which may have a coating weight of about 25-65 grams per square meter (g.s.m.). Thicker adhesives, or combinations of adhesives, may be applied in some embodiments to improve the seal and reduce leaks. Other example embodiments of an attachment device may include a double-sided tape, paste, hydrocolloid, hydrogel, silicone gel, or organogel.

The solution source 145 may also be representative of a container, canister, pouch, bag, or other storage component, which can provide a solution for instillation therapy. Compositions of solutions may vary according to a prescribed therapy, but examples of solutions that may be suitable for some prescriptions include hypochlorite-based solutions, silver nitrate (0.5%), sulfur-based solutions, biguanides, cationic solutions, and isotonic solutions.

In operation, the tissue interface 120 may be placed within, over, on, or otherwise proximate to a tissue site. If the tissue site is a wound, for example, the tissue interface 120 may partially or completely fill the wound, or it may be placed over the wound. The cover 125 may be placed over the tissue interface 120 and sealed to an attachment surface near a tissue site. For example, the cover 125 may be sealed to undamaged epidermis peripheral to a tissue site. Thus, the dressing 110 can provide a sealed therapeutic environment proximate to a tissue site, substantially isolated from the external environment, and the negative-pressure source 105 can reduce pressure in the sealed therapeutic environment.

The process of reducing pressure may be described illustratively herein as “delivering,” “distributing,” or “generating” negative pressure, for example. In general, exudate and other fluid flow toward lower pressure along a fluid path. Thus, the term “downstream” typically implies a location in a fluid path relatively closer to a source of negative pressure or further away from a source of positive pressure. Conversely, the term “upstream” implies a location relatively further away from a source of negative pressure or closer to a source of positive pressure. Similarly, it may be convenient to describe certain features in terms of fluid “inlet” or “outlet” in such a frame of reference. This orientation is generally presumed for purposes of describing various features and components herein. However, the fluid path may also be reversed in some applications, such as by substituting a positive-pressure source for a negative-pressure source, and this descriptive convention should not be construed as a limiting convention.

Negative pressure applied to the tissue site through the tissue interface 120 in the sealed therapeutic environment can induce macro-strain and micro-strain in the tissue site. Negative pressure can also remove exudate and other fluid from a tissue site, which can be collected in canister 115.

In some embodiments, the controller 130 may receive and process data from one or more sensors, such as the first sensor 135. The controller 130 may also control the operation of one or more components of the therapy system 100 to manage the pressure delivered to the tissue interface 120. In some embodiments, controller 130 may include an input for receiving a desired target pressure and may be programmed for processing data relating to the setting and inputting of the target pressure to be applied to the tissue interface 120. In some example embodiments, the target pressure may be a fixed pressure value set by an operator as the target negative pressure desired for therapy at a tissue site and then provided as input to the controller 130. The target pressure may vary from tissue site to tissue site based on the type of tissue forming a tissue site, the type of injury or wound (if any), the medical condition of the patient, and the preference of the attending physician. After selecting a desired target pressure, the controller 130 can operate the negative-pressure source 105 in one or more control modes based on the target pressure and may receive feedback from one or more sensors to maintain the target pressure at the tissue interface 120.

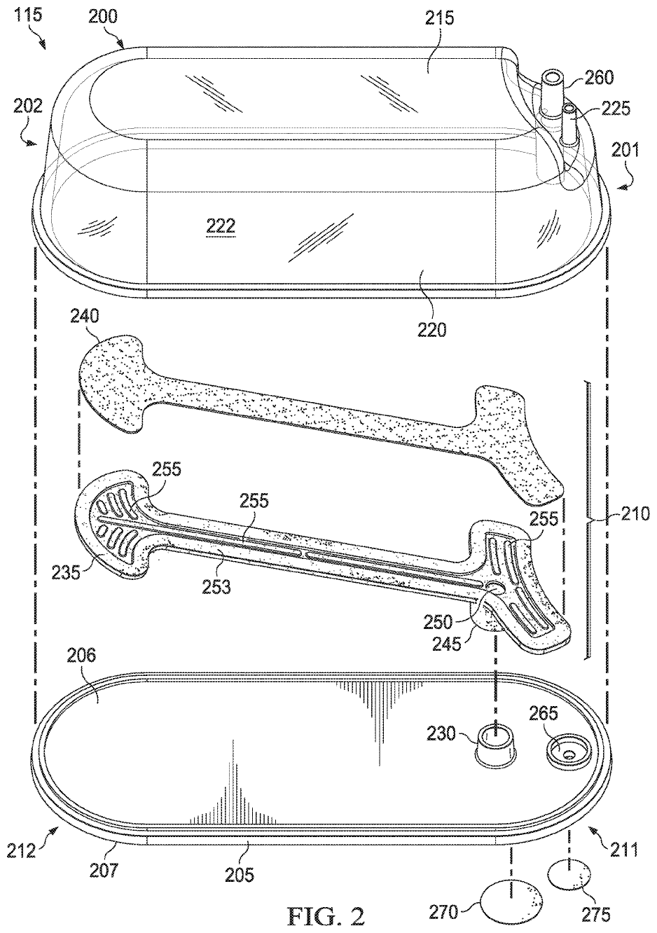

FIG. 2 is an exploded view of the canister 115, illustrating additional details that may be associated with some embodiments. The canister 115 may include a canister body 200, a canister lid 205, and a filter assembly 210. The canister body 200 includes a top portion 215 and at least one wall, such as a side wall 220. The canister body 200 also includes a first body end 201 and a second body end 202 opposite the first body end 201. The canister body 200 is configured to be coupled to the canister lid 205 and define a liquid collection chamber 222 within the canister body 200 and the canister lid 205. The liquid collection chamber 222 may include interior surfaces of the top portion 215 and the side wall 220 of the canister body 200 and an interior surface 206 of the canister lid 205. The liquid collection chamber 222 may be configured to collect liquid from the tissue site. For example, the interior surfaces of the top portion 215 and the side wall 220 of the canister body 200 and the interior surface 206 of the canister lid 205 may be exposed to the liquid collected from the tissue site.

The canister body 200 may also include a collection port 225 configured to fluidly couple the liquid collection chamber 222 to the tissue site. For example, the collection port 225 may fluidly couple the liquid collection chamber 222 and the dressing 110. The collection port 225 may be disposed in the top portion 215 of the canister body 200. In some embodiments, the collection port 225 may be disposed in the top portion 215 adjacent the first body end 201 of the canister body 200. Fluid from the tissue site may be configured to enter the liquid collection chamber 222 through the collection port 225. For example, a conduit or tube may fluidly couple the tissue site and the liquid collection chamber 222 via the collection port 225.

The canister lid 205 includes a first lid end 211 and a second lid end 212 opposite the first lid end 211. In some embodiments, the canister lid 205 includes a reduced pressure port 230. For example, the reduced pressure port 230 may be disposed in the canister lid 205 adjacent the first lid end 211. The reduced pressure port 230 may be configured to fluidly couple the liquid collection chamber 222 to a negative pressure source, such as the negative-pressure source 105.

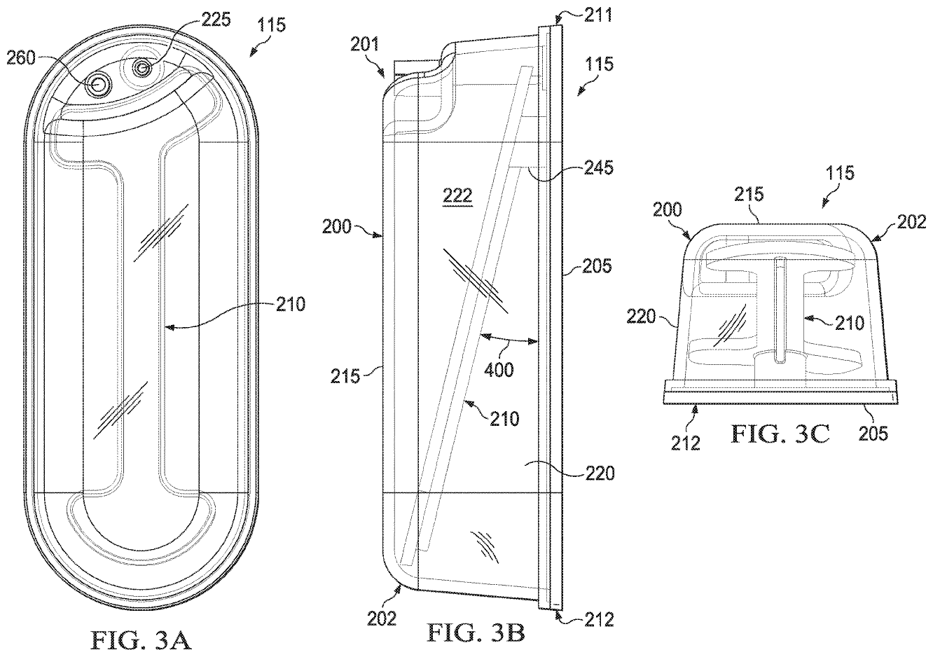

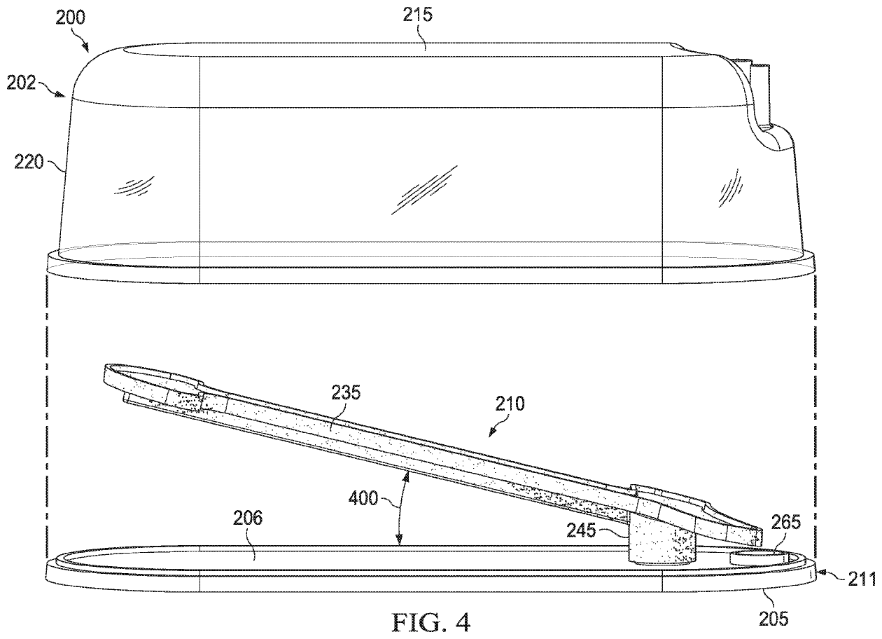

Referring still to FIG. 2, but also FIGS. 3A-4, the filter assembly 210 and the positioning of the filter assembly 210 within the canister 115 are described in more detail. The filter assembly 210 is configured to be disposed in the liquid collection chamber 222 defined by the canister body 200 and the canister lid 205 in fluid communication between the liquid collection chamber 222 and the reduced pressure port 230. The filter assembly 210 may include a filter carrier 235 and a filter 240. In some embodiments, the filter carrier 235 is configured to support the filter 240 and be positioned in fluid communication between the filter 240 and the reduced pressure port 230. The filter carrier 235 may also be configured to be suspended within the liquid collection chamber 222 and separated from the interior walls or surfaces of the canister 115. For example, the filter carrier 235 may be suspended as a cantilever within the liquid collection chamber 222. The filter carrier 235 may be separated from interior surfaces of the top portion 215 and the side wall 220 of the canister body 200 and the interior surface 206 of the canister lid 205.

As shown in FIGS. 2, 3A, and 3B, the filter carrier 235 may also be configured to extend an entire length of the canister 115. Additionally, as shown in FIGS. 2, 3B, 3C, and 4, the filter carrier 235 may be configured to extend the length of the canister 115 at an angle, such as an angle 400, so that at least a portion of the filter carrier 235 or filter assembly 210 reaches opposite and transverse ends of the liquid collection chamber 222 defined by the canister 115. For example, the filter carrier 235 may extend from the first lid end 211 of the canister lid 205 across the side wall 220 towards the top portion 215 and the second body end 202 of the canister body 200. In some embodiments, the angle 400 may be an acute angle less than about 90° relative to the interior surface 206 of the canister lid 205.

In some embodiments, the filter carrier 235 may include a carrier fitting 245 configured to be coupled to at least a portion of the canister lid 205 and suspend the filter carrier 235 in the liquid collection chamber 222 away from the canister lid 205. For example, the carrier fitting 245 may be configured to separate the filter carrier 235 from the interior surfaces of the top portion 215 and the side wall 220 of the canister body 200 and the interior surface 206 of the canister lid 205.

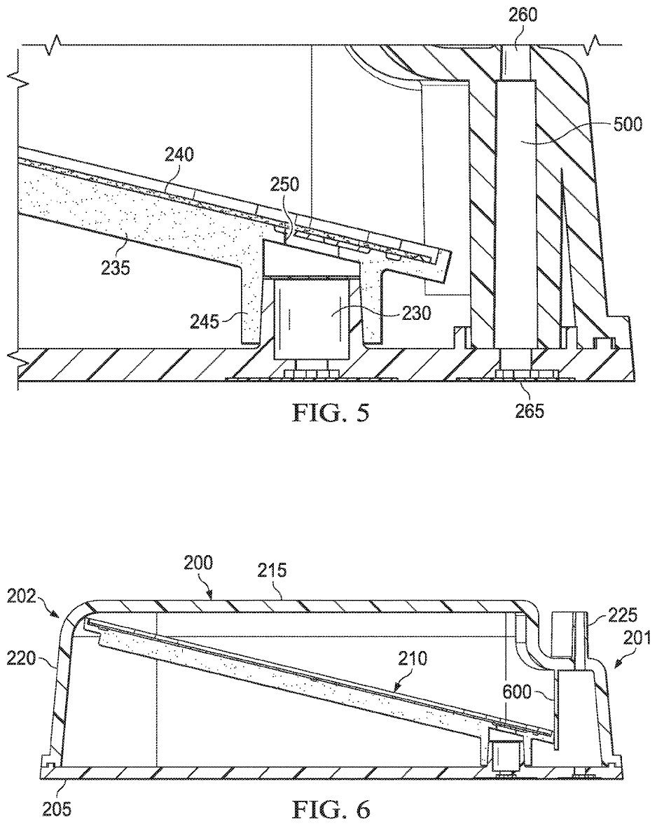

With reference to FIG. 5, the carrier fitting 245 may be configured to be coupled to the reduced pressure port 230 of the canister lid 205 to suspend the filter carrier 235 in the liquid collection chamber 222. In some embodiments, the carrier fitting 245 may be removably coupled to the reduced pressure port 230 by an interface fit. For example, the carrier fitting 245 may be configured to surround an exterior portion of the reduced pressure port 230. The reduced pressure port 230 may suspend the filter carrier 235 within the liquid collection chamber 222 away from interior surfaces of the top portion 215 and the side wall 220 of the canister body 200 and the interior surface 206 of the canister lid 205. The filter carrier 235 and the carrier fitting 245 may include an aperture 250 configured to maintain fluid communication between the negative-pressure source 105 and the liquid collection chamber 222 through the reduced pressure port 230. In other embodiments, a portion of the filter carrier 235, such as the carrier fitting 245, may be adhered to the canister lid 205, such as by an adhesive. In still other embodiments, a portion of the filter carrier 235, such as the carrier fitting 245, may be welded to the canister lid 205.

Referring again to FIG. 2, the filter carrier 235 may have an elongate, planar shape in some embodiments. The filter carrier 235 may also have enlarged portions at one or both ends of the filter carrier 235. For example, the filter carrier 235 may taper from one or both ends towards a center portion of the filter carrier 235. In some embodiments, the filter carrier 235 includes a plurality of ribs or raised portions, such as stand-offs 255, on an upper surface 253 of the filter carrier 235. The plurality of stand-offs 255 may be configured to engage the filter 240 when the filter 240 is coupled to the filter carrier 235. The stand-offs 255 may also be configured to provide a fluid passageway between the filter carrier 235 and the filter 240 to aid with transmission of gases between the reduced pressure port 230 and the liquid collection chamber 222 of the canister 115. In some embodiments, the stand-offs 255 may be thermoformed on the upper surface 253 of the filter carrier 235.

In some embodiments, the filter 240 is sized and shaped to match a size and shape of the filter carrier 235. The filter 240 may be configured to be coupled to the upper surface 253 of the filter carrier 235. The filter 240 may also be configured to permit the transmission of gases, but substantially prevent the transmission of liquids through the filter 240. In some embodiments, the filter 240 may be a liquid-air separator. In other embodiments, the filter 240 may comprise a hydrophobic material to make the filter 240 impermeable to liquid.

In some additional embodiments, the canister 115 may include a first sensing port 260 disposed in the canister body 200 and a second sensing port 265 disposed in the canister lid 205. The first sensing port 260 may be disposed adjacent the first body end 201 of the canister body 200. The first sensing port 260 may also be disposed in the canister body 200 adjacent the collection port 225. In some embodiments the first sensing port 260 is configured to be fluidly coupled to the tissue site. For example, a tube or conduit may fluidly couple the first sensing port 260 to the tissue site or a dressing, such as the dressing 110, positioned at the tissue site.

The second sensing port 265 may be disposed adjacent the first lid end 211 of the canister lid 205. The second sensing port 265 may also be adjacent the reduced pressure port of the canister lid 205 in some embodiments. The second sensing port 265 may be fluidly coupled to the therapy unit 160. In some embodiments, a second filter 270 may be coupled to the reduced pressure port 230. For example, the second filter 270 may be coupled to the exterior surface 207 of the canister lid 205 adjacent the reduced pressure port 230. The second filter 270 may be gas permeable and liquid impermeable to permit the transmission of gases, but substantially prevent the transmission of liquids through the reduced pressure port 230. Additionally, a third filter 275 may be fluidly coupled to the second sensing port 265 in some embodiments. For example, the third filter 275 may be coupled to the exterior surface 207 of the canister lid 205 adjacent the second sensing port 265. The third filter 275 may also be gas permeable and liquid impermeable to permit the transmission of gases, but substantially prevent the transmission of liquids through the second sensing port 265.

Referring again to FIG. 5, the first sensing port 260 and the second sensing port 265 may be fluidly coupled and form a sensing pathway 500. In some embodiments, the sensing pathway 500 may be formed by or may be a tube or conduit fluidly coupling the first sensing port 260 and the second sensing port 265. The sensing pathway 500 may be fluidly isolated from the liquid collection chamber 222 of the canister 115.

In operation, reduced pressure from the negative-pressure source 105 may be communicated to the tissue site through the reduced pressure port 230, the liquid collection chamber 222, and the collection port 225 of the canister 115. When the reduced pressure is applied from the negative-pressure source 105 to the canister 115, the reduced pressure is delivered to the tissue site, which results in liquid at the tissue site being drawn into the liquid collection chamber 222 of the canister 115 through the collection port 225. Liquid begins to fill the canister 115 but is substantially prevented from passing through or blocking the reduced pressure port 230 by the filter 240. As long as a portion of the filter assembly 210 remains uncovered by liquid, the filter carrier 235 and the filter 240 of the filter assembly 210 will continue to permit gas flow and transmission of reduced pressure.

Additionally, the sensing pathway 500 formed by fluidly coupling the first sensing port 260 and the second sensing port 265 may monitor the reduced pressure delivered to the tissue site. For example, the sensing pathway 500 may be coupled to the first sensor 135 of the therapy unit 160 and configured to measure negative pressure at the dressing 110. A controller, such as the controller 130 of FIG. 1, can operate a negative-pressure source, such as the negative-pressure source 105 of FIG. 1, based on input from the first sensor 135 to provide therapeutic levels of negative pressure to the dressing 110.

FIG. 6 is a cross-section view of the canister 115 of FIG. 2, illustrating additional details that may be associated with some embodiments. In some embodiments, the canister body 200 may include a fluid shield 600 adjacent the collection port 225. For example, the fluid shield 600 may be coupled to an interior surface of the top portion 215 of the canister body 200 and be positioned between the collection port 225 and the filter 240. The fluid shield 600 may extend at least partially into the collection chamber toward the canister lid 205. In some embodiments, the fluid shield 600 is configured to prevent fluid from coming into direct contact the filter 240 of the filter assembly 210 as the fluid enters the liquid collection chamber 222 through the collection port 225.

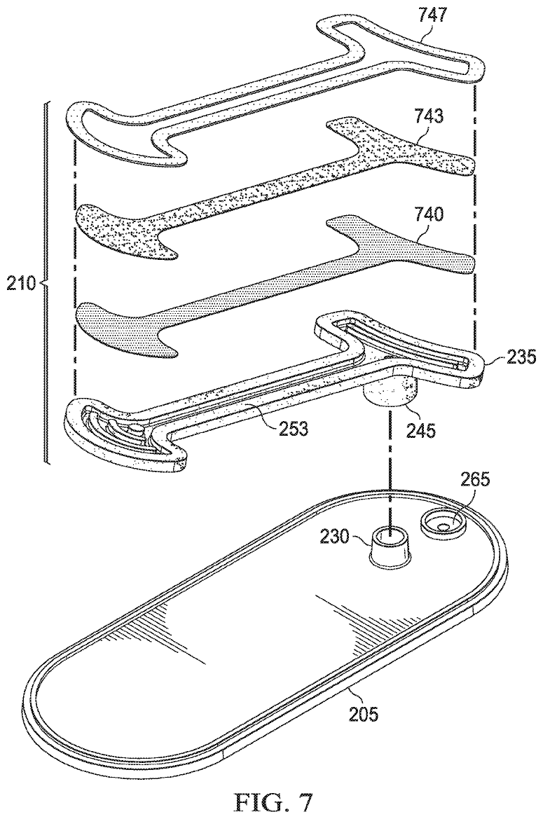

FIG. 7 is an exploded view of another embodiment of the filter assembly 210, illustrating additional details that may be associated with some embodiments. In some embodiments, the filter assembly 210 may include the filter carrier 235 and one or more filters. For example, the filter assembly 210 may include a first filter 740 and a second filter 743. The first filter 740 may be similar or identical to the filter 240, discussed above. In some embodiments, the second filter 743 may comprise an odor filter. In some additional embodiments, the second filter 743 may comprise a charcoal filter. The filter assembly 210 may also include an adhesive 747 configured to couple the first filter 740 and the second filter 743 to the filter carrier 235. For example, at least a portion of the first filter 740 may be coupled to the upper surface 253 of the filter carrier 235, and the second filter 743 may be coupled adjacent the first filter 740. The adhesive 747 may be configured to cover at least a perimeter of the second filter 743 to couple the second filter 743 and the first filter 740 to at least a portion the filter carrier 235.

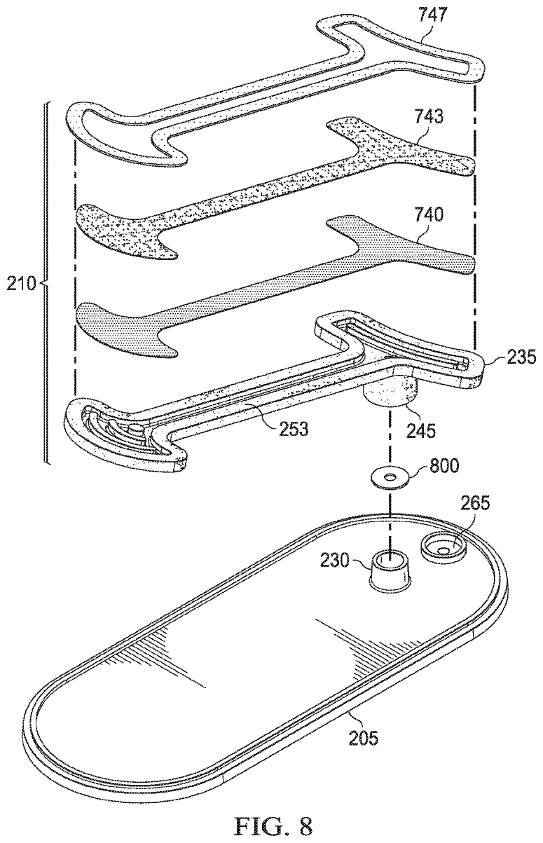

FIG. 8 is an exploded view of another embodiment of the filter assembly 210, illustrating additional details that may be associated with some embodiments. Similar to FIG. 7, the filter assembly 210 may include the filter carrier 235, the first filter 740, the second filter 743, and the adhesive 747. In some embodiments, the filter carrier 235 may be formed from a heat formed closed-cell foam. In other embodiments, the filter carrier 235 may be formed from an extruded closed-cell foam. Additionally, the filter assembly 210 may include a gasket or sealing ring, such as a sealing ring 800, positioned between the carrier fitting 245 of the filter carrier 235 and the reduced pressure port 230. The sealing ring 800 may be configured to provide a fluid seal between the carrier fitting 245 and the reduced pressure port 230.

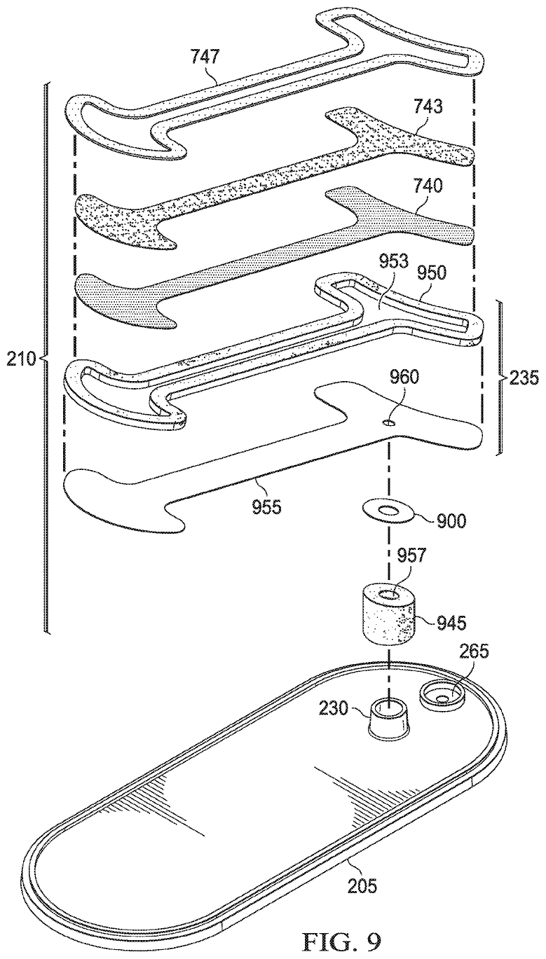

FIG. 9 is an exploded view of another embodiment of the filter assembly 210, illustrating additional details that may be associated with some embodiments. Similar to FIG. 7, the filter assembly 210 may include the filter carrier 235, the first filter 740, the second filter 743, and the adhesive 747. In some embodiments, the filter carrier 235 comprises a foam layer 950 and a film layer 955. The foam layer 650 may include a hollow interior portion 953. In some embodiments, the foam layer 950 and the film layer 955 may be coupled to form the filter carrier 235. The first filter 740 and the second filter 743 may be configured to be coupled to the foam layer 950 of the filter carrier 235 by the adhesive 747

In some embodiments, the filter carrier 235 is configured to be coupled to the reduced pressure port 230 by a carrier fitting 945. In some embodiments, the carrier fitting 945 may be removably coupled to the reduced pressure port 230 by an interference fit. For example, the carrier fitting 945 may include an aperture 957 configured to surround an exterior portion of the reduced pressure port 230. The aperture 957 may be configured to fluidly couple the reduced pressure port 230 to the hollow interior portion 953 of the filter carrier 235 via an aperture 960 in the film layer 955.

Additionally, the filter assembly 210 may include the sealing ring 900 in some embodiments. The sealing ring 900 may be configured to create a fluid seal between the carrier fitting 945 and the film layer 955. The sealing ring 900 may also be configured to surround the aperture 960 in the film layer 955 and the aperture 957 in the carrier fitting 945.

With reference to FIGS. 10-12B, a filter assembly 1010 that may be associated with some embodiments of the canister 115 is illustrated. In some embodiments, the filter assembly 1010 may have a cross shape as shown in FIG. 10. In such embodiments, the filter assembly 1010 may include a filter carrier 1015 and a filter 1020 configured to be coupled to the filter carrier 1015. In other embodiments, the filter assembly 1010 may include a plurality of filters coupled to the filter carrier 1015.

In some embodiments, the filter assembly 1010 may have a spherical shape, as shown in FIGS. 11A and 11B. The filter assembly 1010 may also include an aperture or a recess in an exterior portion of the filter assembly 1010. For example, the filter assembly 1010 may include an aperture 1150 as shown in FIG. 11B. The filter assembly 1010 may also include a sintered polymer in some embodiments.

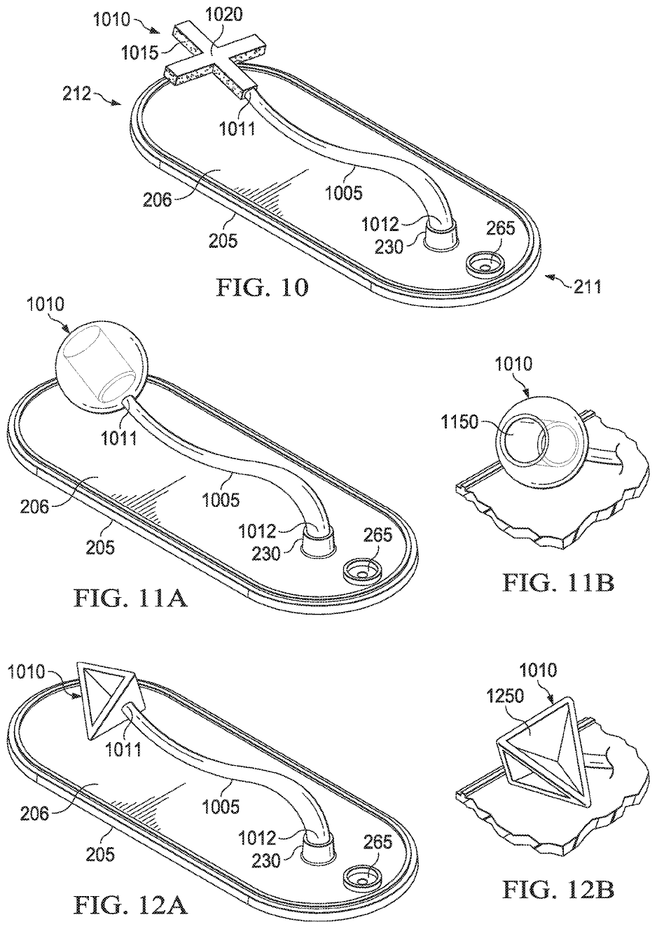

In some additional embodiments, the filter assembly 1010 may have a pyramidal shape, as illustrated in FIGS. 12A and 12B. In such embodiments, the filter assembly 1010 may include a sintered polymer. The filter assembly 1010 may also include an aperture or a recess in an exterior portion of the filter assembly 1010. For example, the filter assembly 1010 may include one or more recesses 1250 as shown in FIGS. 12A and 12B.

Referring still to FIGS. 10-12B, the filter assembly 1010 may include a conduit, such as a flexible tube 1005, configured to couple the filter assembly 1010 to the canister lid 205. In some embodiments, the flexible tube 1005 includes a first end 1011 and a second end 1012. The first end 1011 may be configured to be coupled to the filter assembly 1010 and the second end 1012 may be configured to be coupled to at least a portion of the interior surface 206 of the canister lid 205. For example, the second end 1012 of the flexible tube 1005 may be coupled to a portion of the interior surface 206 of the canister lid 205 adjacent the first lid end 211. Additionally or alternatively, the second end 1012 of the flexible tube 1005 may be fluidly coupled to the reduced pressure port 230 in the canister lid 205. For example, the flexible tube 1005 may be configured to fluidly couple the reduced pressure port 230 to the filter assembly 1010. In other embodiments, the second end 1012 of the flexible tube 1005 may be coupled to a portion of the interior surface 206 of the canister lid 205 adjacent the second lid end 212. In still other embodiments, the reduced pressure port 230 may be disposed in the canister lid 205 adjacent the second lid end 212. In such embodiments, the second end 1012 of the flexible tube may still be fluidly coupled to the reduced pressure port 230.

In some embodiments, the flexible tube 1005 is configured to allow the filter assembly 1010 to move within the collection chamber of the canister 115. For example, the filter assembly 1010 may be buoyant and configured to be moveable within and from a first end of the liquid collection chamber 222 to a second end of the liquid collection chamber 222. In such embodiments, the filter assembly 1010 is configured to remain above a fluid level within the liquid collection chamber 222 of the canister 115. For example, the filter assembly 1010 is configured to float within the liquid collection chamber 222 in order to permit gas flow and transmission of reduced pressure regardless of the orientation of the canister 115.

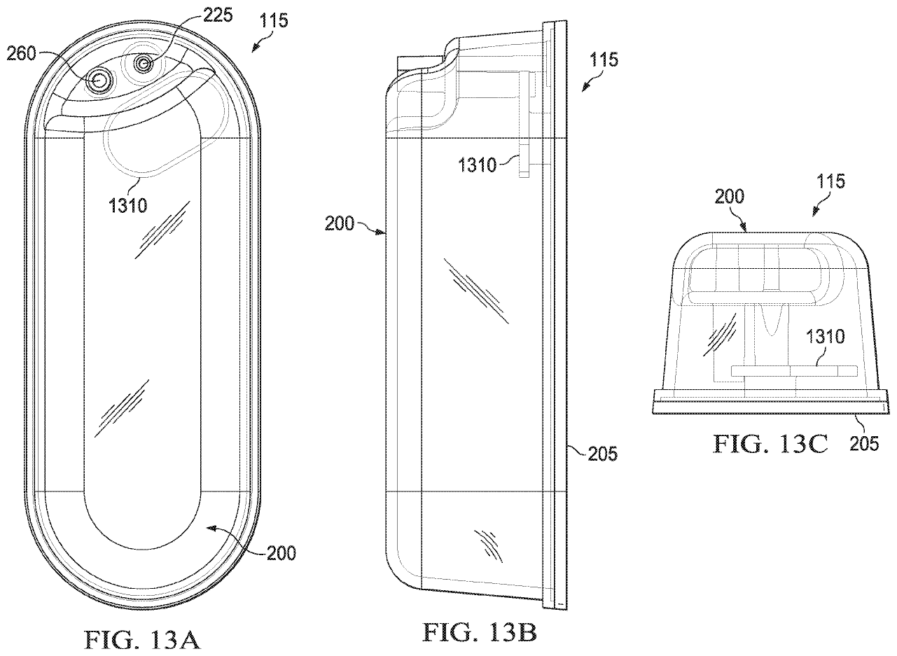

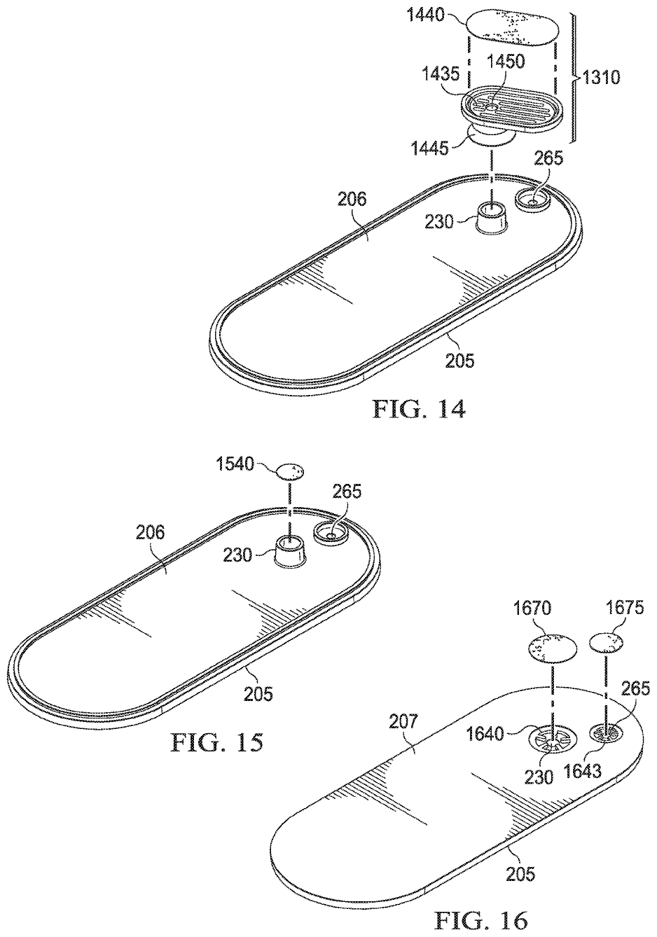

Referring to FIGS. 13A-13C, another embodiment of the canister 115 is illustrated. In some embodiments, the canister 115 may include a single-orientation filter assembly, such as filter assembly 1310. The filter assembly 1310 may be positioned within the canister 115 in a similar manner as the filter assembly 210. In some embodiments, the filter assembly 1310 may be configured to be coupled to at least a portion of the canister lid 205. For example, the filter assembly 1310 may be configured to be coupled to the reduced pressure port 230, as shown in FIG. 14.

Referring still to FIGS. 13A-13C, but also FIG. 14, the filter assembly 1310 may include a filter carrier 1435 and a filter 1440, similar to the filter assembly 210. In some embodiments, the filter carrier 1435 includes a carrier fitting 1445. The carrier fitting 1445 may be configured to couple the filter carrier 1435 to the reduced pressure port 230 of the canister lid 205. In some embodiments, an aperture 1450 may be disposed in the filter carrier 1435. The aperture 1450 may be fluidly coupled to the reduced pressure port 230 via a corresponding aperture in the carrier fitting 1445.

In some embodiments, the filter 1440 is configured to be coupled to the filter carrier 1435. The filter 1440 may be gas permeable and liquid impermeable to permit the transmission of gases, but substantially prevent the transmission of liquids. For example, the filter 1440 may permit the transmission of gasses through the reduced pressure port 230 and the aperture 1450 while preventing liquid from passing through the aperture 1450 and the reduced pressure port 230. In other embodiments, the filter 1440 may include a plurality of filters configured to be coupled to the filter carrier 1435.

FIG. 15 is an exploded view of another embodiment of the canister lid 205 of the canister 115, illustrating additional that may be associated with some embodiments. In some embodiments, the canister 115 may include a filter 1540. The filter 1540 may be positioned within the reduced pressure port 230 of the canister lid 205. The filter 1540 may be configured to prevent liquid from exiting the liquid collection chamber 222 of the canister 115 through the reduced pressure port 230 while also allowing gaseous communication between the liquid collection chamber 222 and the reduced pressure port 230.

FIG. 16 is an exploded view of yet another embodiment of the canister lid 205 of the canister 115, illustrating additional features that may be associated with some embodiments. In some embodiments, the canister lid 205 may include one or more welded or sintered filters disposed in the canister lid 205. For example, the canister lid 205 may include a first filter 1640 and a second filter 1643. In some embodiments, the first filter 1640 may be associated with the reduced pressure port 230 and the second filter 1643 may be associated with the second sensing port 265.

Additionally or alternatively, the canister lid 205 may include one or more filters coupled to at least a portion of the exterior surface 207. For example, a third filter 1670 and a fourth filter 1675 may be coupled to the exterior surface 207 of the canister lid 205. The third filter 1670 and the fourth filter 1675 may be similar to the second filter 270 and the third filter 275, respectively, as shown in FIG. 2. In some embodiments, the third filter 1670 is configured to be coupled adjacent the first filter 1640 and the fourth filter 1675 is configured to be coupled adjacent the second filter 1643.

A method of manufacturing an apparatus for managing fluid from a tissue site is also disclosed. In some embodiments, the method includes providing a canister lid, providing a filter, coupling a filter to the filter carrier, and coupling the filter carrier to a portion of the canister lid. In some embodiments, coupling the filter carrier to a portion of the canister lid comprises welding the filter carrier to the canister lid. In some embodiments, coupling the filter to the filter carrier comprises coupling the filter to the filter carrier with an adhesive. In some additional embodiments, the method may further comprise coupling an odor filter and/or a charcoal filter to the filter carrier

The method further includes providing a canister body and coupling the canister lid to the canister body. In some embodiments, the canister body and the canister lid define a liquid collection chamber, and the filter carrier and the filter may be positioned within the liquid collection chamber. The method may also comprise forming a collection port in the canister body. The collection port may be configured to fluidly couple the liquid collection chamber to the tissue site.

The method may further comprise forming a reduced pressure port in the canister lid. The reduced pressure port may be configured to be fluidly coupled to a reduced pressure source. In some embodiment, the method additionally includes coupling a second filter to the reduced pressure port. The second filter may be coupled to an exterior portion of the canister lid adjacent the reduced pressure port.

Additionally, the method may comprise forming a first sensing port in the canister body and forming a second sensing port in the canister lid. The first sensing port and the second sensing port may be configured to be fluidly coupled. The method may also include fluidly coupling the first sensing port to a tissue site and fluidly coupling the second sensing port to a reduced pressure source. The method may further comprise coupling a third filter to the second sensing port. The third filter may be coupled an exterior portion of the canister lid adjacent the second sensing port.

The systems, apparatuses, and methods described herein may provide significant advantages. For example, the embodiments described herein may provide a canister 115 that may deliver reduced pressure to a tissue site and collect fluid from the tissue site when positioned in multiple orientations, which improves user experience and allows more of the internal volume of the canister 115 to be used. Because the canister 115 does not need to be positioned in a predetermined orientation, the canister 115 can be carried discreetly, such as placed within a patient's bag without having to be positioned in a specific orientation. The canister 115 also reduces the number of components within the canister so more of the internal volume may be utilized. For example, a single filter assembly 210 is positioned within the canister 115 and reaches the extremities of the canister 115 in order to improve fill capacity without changing the overall size of the canister 115.

Additionally, some embodiments of the canister 115 described herein may be modified to be used in a single orientation, which may simplify and reduce the costs of manufacturing the canister 115. For example, the canister 115 may be manufactured from a single set of core parts allowing for use in a single orientation or multiple orientations. This provides greater flexibility to use the canister 115 to suit a patient or clinician's particular needs.

While shown in a few illustrative embodiments, a person having ordinary skill in the art will recognize that the systems, apparatuses, and methods described herein are susceptible to various changes and modifications that fall within the scope of the appended claims. Moreover, descriptions of various alternatives using terms such as “or” do not require mutual exclusivity unless clearly required by the context, and the indefinite articles “a” or “an” do not limit the subject to a single instance unless clearly required by the context. Components may also be combined or eliminated in various configurations for purposes of sale, manufacture, assembly, or use. For example, in some configurations the dressing 110, the canister 115, or both may be eliminated or separated from other components for manufacture or sale. In other example configurations, the controller 130 may also be manufactured, configured, assembled, or sold independently of other components.

The appended claims set forth novel and inventive aspects of the subject matter described above, but the claims may also encompass additional subject matter not specifically recited in detail. For example, certain features, elements, or aspects may be omitted from the claims if not necessary to distinguish the novel and inventive features from what is already known to a person having ordinary skill in the art. Features, elements, and aspects described in the context of some embodiments may also be omitted, combined, or replaced by alternative features serving the same, equivalent, or similar purpose without departing from the scope of the invention defined by the appended claims.

1. A liquid-collection canister for collecting liquid from a tissue site to which reduced pressure treatment is applied, the canister comprising:
a canister body;
a canister lid configured to be coupled to the canister body and to define a liquid collection chamber with the canister body, the liquid collection chamber including one or more internal walls configured to collect and to be exposed to the liquid from the tissue site;
a filter carrier suspended within the liquid collection chamber and separated from the internal walls; and
a filter coupled to the filter carrier, the filter configured to allow gaseous communication with the liquid collection chamber and to prevent liquid from leaving the liquid collection chamber.

2. The liquid-collection canister of claim 1, further comprising a reduced pressure port in fluid communication with the liquid collection chamber, wherein the filter carrier is configured to be fluidly coupled to the reduced pressure port.

3. The liquid-collection canister of claim 2, wherein the reduced pressure port is disposed in the canister lid and configured to fluidly couple the liquid collection chamber to a negative pressure source.

4. (canceled)

5. The liquid-collection canister of claim 2, wherein the filter carrier is configured to support the filter and to be positioned in fluid communication between the filter and the reduced pressure port.

6. The liquid-collection canister of claim 2, wherein the filter carrier is suspended in the liquid collection chamber from the reduced pressure port.

7. The liquid-collection canister of claim 6, further comprising a carrier fitting configured to fluidly couple the filter carrier to the reduced pressure port, wherein the carrier fitting is removably coupled to the reduced pressure port by an interference fit.

8. The liquid-collection canister of claim 6, wherein the filter carrier is suspended as a cantilever within the liquid collection chamber, and wherein the filter carrier and the filter are not coupled to the internal walls of the liquid collection chamber.

9. The liquid-collection canister of claim 1, further comprising a collection port configured to fluidly couple the liquid collection chamber to the tissue site.

10. (canceled)

11. (canceled)

12. (canceled)

13. (canceled)

14. The liquid-collection canister of claim 1, wherein the filter carrier comprises a planar shape configured to extend from a first end of the liquid collection chamber to a second end of the liquid collection chamber.

15. The liquid-collection canister of claim 14, wherein the filter carrier is configured to extend from the first end to the second end at an angle.

16. (canceled)

17. The liquid-collection canister of claim 1, wherein a flexible tube is configured to couple the filter carrier to a reduced pressure port in fluid communication with the liquid collection chamber such that the filter carrier and the filter are moveable within and from a first end of the liquid collection chamber to a second end of the liquid collection chamber.

18. (canceled)

19. (canceled)

20. (canceled)

21. (canceled)

22. A liquid-collection canister for collecting liquid from a tissue site, comprising:
a liquid collection chamber configured to collect the liquid from the tissue site; and
a filter assembly configured to be positioned within the liquid collection chamber in fluid communication between the liquid collection chamber and a reduced
pressure port, wherein at least a portion of the filter assembly is configured to reach opposite and transverse ends of the liquid collection chamber.

23. The liquid-collection canister of claim 22, wherein the filter assembly comprises a filter carrier and a filter coupled to the filter carrier.

24. (canceled)

25. (canceled)

26. The liquid-collection canister of claim 23, wherein the filter is a gas permeable and liquid impermeable filter, the filter assembly further comprising a charcoal filter coupled to the filter carrier.

27. The liquid-collection canister of claim 23, wherein the filter carrier comprises a thermoformed closed-cell foam.

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. The liquid-collection canister of claim 22, wherein the filter assembly is suspended in the liquid collection chamber from the reduced pressure port.

40. The liquid-collection canister of claim 39, further comprising a carrier fitting configured to fluidly couple the filter assembly to the reduced pressure port, wherein the carrier fitting is removably coupled to the reduced pressure port by an interference fit.

41. The liquid-collection canister of claim 39, wherein the filter assembly is suspended as a cantilever within the liquid collection chamber, and wherein the filter assembly is not coupled to internal walls of the liquid collection chamber.

42. A system for collecting liquid from a tissue site to which reduced pressure treatment is applied, the system comprising:
a liquid collection canister, comprising:
a canister body comprising a collection port and a first sensing port;
a canister lid configured to define a liquid collection chamber relative to the canister body, the canister lid comprising a reduced pressure port and a second sensing port, the first second port configured to be fluidly coupled to the first sensing port;
a filter carrier configured to be coupled to the reduced pressure port and to extend across the liquid collection chamber; and
a filter coupled to the filter carrier, the filter configured to allow gaseous communication and prevent liquid from leaving the canister body;
a dressing fluidly coupled to the collection port and the first sensing port; and
a reduced pressure source fluidly coupled to the reduced pressure port.

43. The system of claim 42, wherein the filter carrier is configured to extend from the canister lid to the canister body at an angle.

44. (canceled)

45. The system of claim 42, wherein the filter carrier comprises an elongate body and a fitting, the fitting configured to suspend the elongate body in the liquid collection chamber and to fluidly couple the elongate body to the reduced pressure port.

46. (canceled)
